(19) United States
(12) Patent Application Publication
(10) Pub. No.: US 2024/0157111 A1
(43) Pub. Date: May 16, 2024

(54) SELF FLUSHING VALVE

(71) Applicant: CareFusion 303, Inc., San Diego, CA (US)

(72) Inventor: Raymond P. FEITH, Chino Hills, CA (US)

(21) Appl. No.: 17/987,119

(22) Filed: Nov. 15, 2022

(51) Int. Cl. A61M 39/22; A61M 39/10; F16L 37/46; F16L 58/18

(52) U.S. Cl. CPC A61M 39/225; A61M 39/10; F16L 37/46; F16L 58/185

(57) ABSTRACT

Connector assemblies are disclosed. A connector assembly includes a housing with a post and a valve surrounds the post. The valve is displaceable and based upon the displacement allows fluid to pass therethrough. While providing fluid, a fluid delivery device provides an external force to displace the valve, causing the valve to form a fluid pocket that receives fluid from the fluid delivery device. As the fluid delivery device is removed from the connector assembly, the fluid pocket collapses and drives fluid stored in the fluid pocket through an opening in the post, where the fluid flows downstream to a catheter line. Additionally, the valve closes and covers the post prior to removal of the fluid delivery device. The collapsing fluid pocket and the valve closing prevent the fluid previously in the fluid pocket from upstream fluid flow otherwise caused by removal of the fluid delivery device.

### TECHNICAL FIELD

The present disclosure relates generally to medical fluid valves and, more particularly, to valves modified to control medical fluid, including residual medical fluid, provided by syringes. As a syringe is removed from a valve, the valve can drive medical fluid located in a fluid pocket of the valve downstream through the valve and to a catheter.

### BACKGROUND

For an intravenous (“IV”) application, valves, including needleless access valves, can be used to deliver a fluid to a patient. When a syringe provides sufficient pressure to a valve, the valve opens and permits downstream fluid delivery from the syringe to a catheter. However, as the syringe is removed from the valve, some of the remaining fluid can flow upstream due to removal of the syringe, leading to issues such as blood coagulation and catheter blockage or obstruction. When these types of issues occur, the IV set needs to be replaced, leading to added costs and a negative patient experience.

### SUMMARY

Aspects of the present disclosure provide a connector assembly with a post (e.g., center post) and a valve that is positioned on the post. When engaged with a fluid delivery device (e.g., syringe), the valve is displaced (e.g., compressed) relative to the post, causing the post to protrude through the valve. Based on the displacement of the valve, the valve forms a fluid pocket that, in some instances, receives residual fluid from the fluid delivery device. As the fluid delivery device is removed from the valve, the valve begins to return to its original shape, which causes the fluid pocket to collapse. The collapsing of the fluid pocket drives the residual fluid within the fluid pocket downstream through the valve and to a catheter connected to the connector assembly.

Additionally, as the fluid delivery device is removed from the connector assembly, the force provided by the fluid delivery devices reduces, allowing the valve to cover the post. When the post no longer protrudes through the valve, the valve prevents upstream flow of fluid, including the residual fluid previously located in the fluid pocket. Accordingly, the valve promotes downstream flow of the fluid and prevents upstream flow of the fluid. As a result, issues such as blood coagulation and catheter blockage.

In accordance with at least some embodiments disclosed herein is the realization that unintended catheter blockage or obstruction can result in injury to a patient or a caregiver, such as by depriving the patient of a medicament, increasing the potential for infection to the patient, and exposing the caregiver to harmful medicaments. Additionally, unintended catheter blockage can render the IV set unusable.

Accordingly, aspects of the present disclosure provide a connector assembly for use with a catheter, the connector assembly comprising a first housing comprising a fluid inlet, a second housing coupled with the first housing, the second housing comprising a post that includes an opening, and a valve that surrounds post, wherein responsive to an external force, the valve comprises a fluid pocket, and when the external force is removed, the fluid pocket collapses and causes a fluid received from the fluid inlet and stored in the fluid pocket to enter the post through the opening.

Some instances of the present disclosure provide a valve comprising a compressible body, wherein responsive to an external force provided by a fluid delivery device, the compressible body reduces from a first dimension to a second dimension and forms a fluid pocket that receives a fluid from the fluid delivery device, and when the external force is removed, the compressible body increases from the second dimension to the first dimension and causes a volume reduction of the fluid pocket, the volume reduction causing the fluid to exit the fluid pocket.

Some instances of the present disclosure provide a method for regulating a fluid to a catheter, the method comprising, by a valve receiving, at the valve, an external force, forming, based on the external force, a fluid pocket in the valve, receiving, at the fluid pocket, the fluid, when the external force is removed, reducing a volume of the fluid pocket to define a volume reduction, and removing, based on the volume reduction, at least some of the fluid from the fluid pocket.

Accordingly, the present application addresses several operational challenges encountered in prior valves, including needleless access valves, used in administering fluid using a syringe and provides numerous enhancements and improvements for flushing fluid through a catheter, which can reduce fluid backflow and limit or prevent closing or blocking of the catheter.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a perspective view of a connector, in accordance with some aspects of the present disclosure.

FIG. 2 illustrates the connector assembly in FIG. 1 in use with a fluid delivery device, in accordance with some aspects of the present disclosure.

FIGS. 3 and 4 illustrate partial cross-sectional views of the connector assembly, in accordance with some aspects of the present disclosure.

FIGS. 5 and 6 illustrate partial cross-sectional views of the connector assembly, further showing a fluid delivery device inserted into the housing and engaging the valve, in accordance with some aspects of the present disclosure

FIGS. 7 and 8 illustrate partial cross-sectional views of the connector assembly, showing the fluid delivery device further inserted into the housing and further displacing the valve, in accordance with some aspects of the present disclosure.

FIG. 9 illustrates a partial cross-sectional view of the connector assembly, with the fluid delivery device removed from the housing, in accordance with some aspects of the present disclosure.

FIG. 10 illustrates a flowchart showing a method for regulating a fluid to a catheter, in accordance with some aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Needle-free connectors are essential devices to deliver fluid to a patient via an IV catheter. Needle-free connectors may be used in general patient populations, including neonatal, pediatric, and adult patients. In various applications, the pressure applied to the blood component should not exceed 300 mm Hg (5 psi) as this may result in hemolysis or bag breakage, the IV fluid needs to be injected in bolus without control during power injection and infusion pressure should never exceed 25 psi, as pressure higher than 25 psi may damage blood vessels. Thus, medical professional using needle-free connectors face challenges to maintain the various higher-pressure limits during infusion delivery with typical connectors.

Typical needle-free connectors used with syringes have other drawbacks. For example, when the syringed is removed after fluid delivery, a valve located in the needle-free connector can pull at least some fluid back from the catheter, causing blood coagulation and/or catheter blockage. Either issue may result in full replacement of the IV set. The following devices and methods provide design modifications to overcome the foregoing issues.

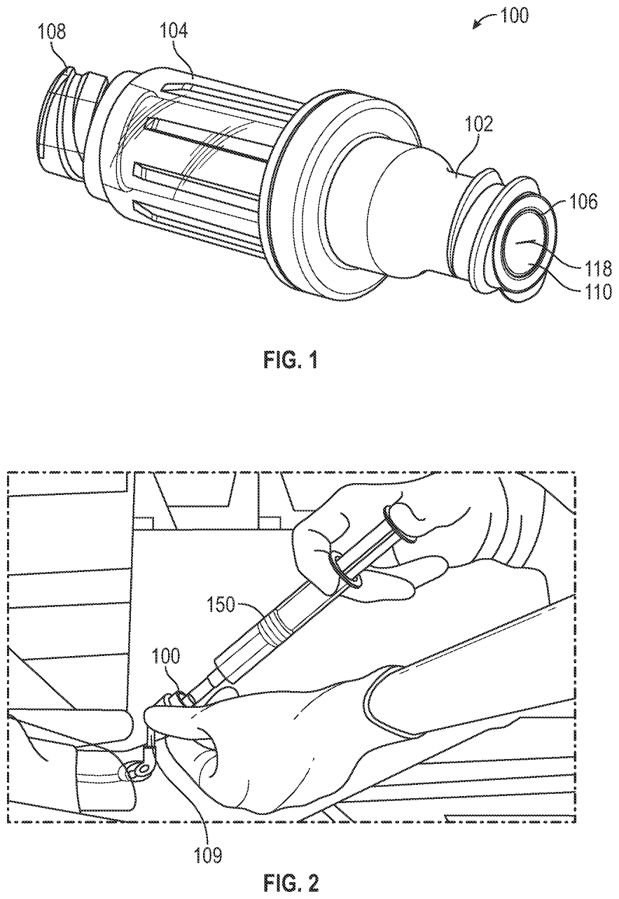

Referring now to the figures, FIGS. 1 and 2 illustrate a perspective view of a connector assembly 100, in accordance with some aspects of the present disclosure. The connector assembly 100 may include a housing 102 and a housing 104 coupled with the housing 102. The housing 102 and the housing 104 may be referred to as a first housing and a second housing, respectively. The coupling between the housing 102 and the housing 104 may be a removable connection such that the housing 102 can be separated from housing 104. The housing 102 includes a fluid inlet 106, and the housing 104 includes a fluid outlet 108. As shown, the fluid inlet 106 and the fluid outlet 108 are cylindrical, or generally cylindrical, bodies with circular cross sections. However, other shapes are possible. To regulate the fluid, the connector assembly 100 includes a valve 110 carried within portions of the housing 102 and the housing 104.

As shown in FIG. 2, the connector assembly 100 may be used to provide a connection point for a fluid delivery device 150. In some embodiments, the fluid delivery device 150 includes a syringe, such as a needle-free syringe. Accordingly, in some embodiments, the connector assembly 100 can take the form of a needle-free connector assembly. For some exemplary IV applications, the fluid delivery device 150 may be used for rapid injections of medication, sometimes referred to as a “push” or “bolus” to quickly send a one-time dose of medication into a patient's bloodstream. The fluid delivery device 150 is connected to the fluid inlet 106 (shown in FIG. 1) of the connector assembly 100. Additionally, the fluid outlet 108 of the connector assembly 100 can be connected to a catheter line 109. The valve 110 is positioned to regulate the flow of fluid provided by the fluid delivery device 150 to the catheter line 109.

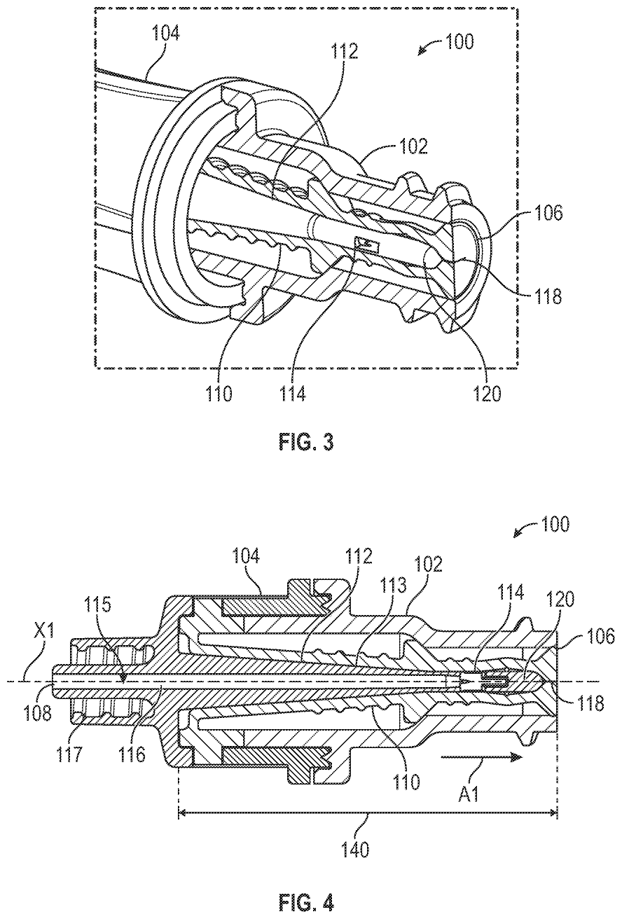

FIGS. 3 and 4 illustrate partial cross-sectional views of the connector assembly 100, in accordance with some aspects of the present disclosure. The housing 104 may include a post 112 that extends into the housing 102. The post 112 provides a hollow body to facilitate the transfer of fluid through the connector assembly 100. The post 112 includes a proximal end and a distal end 120, and, in some embodiments of the present disclosure, an outer surface of the post defines a cross-sectional width that decreases in a direction from the proximal end toward the distal end 120 of the post.

The valve 110 includes a channel 113 that allows the valve 110 to receive the post 112. As shown in FIGS. 3 and 4, the valve 110 surrounds the post 112. In some embodiments, the valve 110 is disposed on, and engaged with, the post 112. Also, the post 112 includes an opening 114 (representative of one or more openings in the post 112) designed to receive fluid from the fluid inlet 106 of the housing 102. Accordingly, the fluid inlet 106 is fluidly connected to the opening 114. For purposes of illustration, the post 112 in FIG. 4 is rotated such that the opening 114 is viewable. As shown in FIG. 4, a lumen 116 is formed by or positioned in a channel 115 of the post 112. The fluid, when received from the opening 114, can subsequently flow into the lumen 116. Accordingly, the opening 114 is fluidly connected to the lumen 116. In order to secure the lumen 116, the housing 104 includes a luer 117.

Based on the position of the valve 110 shown in FIGS. 3 and 4, the valve 110 is in a closed position and covers the opening 114, thereby preventing the flow of fluid into the opening 114 of the post 112 and to the lumen 116. However, the valve 110 is designed to regulate flow based in part upon the displacement (e.g., compression) of the valve 110 such that the opening 114 is uncovered by the valve 110, as will be shown and described in detail below. It should be noted that displacement of the valve 110 may include an elastic displacement (e.g., elastic compression), thus allowing the valve 110 to return, after displacement, to its original form (shown in FIGS. 3 and 4) when a fluid delivery device is removed/disengaged from the valve 110.

Also, a slit 118, representing a cut or other discontinuity in the valve 110, is formed in the valve 110. In the closed position of the valve 110, no object(s) is/are positioned in the slit 118 of the valve 110 and the slit 118 is generally closed. For example, a distal end 120 of the post 112 does not protrude through the slit 118 in the closed position of the valve 110.

Referring to FIG. 4, a longitudinal axis X1 represents an axis that runs parallel to a major dimension of the connector assembly 100. The housing 102 of the connector assembly 100 includes a size such that when the housing 102 is coupled with the housing 104 (as shown in FIG. 4), the housing 102 extends further along the longitudinal axis X1, in the direction of the arrow μl of the longitudinal axis X1, than the post 112. Put another way, the distal end 120 of the post 112 is contained within the housing 102 and does not protrude out of the fluid inlet 106 of the housing 102. Also, when no external forces are acting on the valve 110 and the valve 110 is in the closed position, the valve 110 includes a dimension 140 that represents a lengthwise dimension of the valve 110.

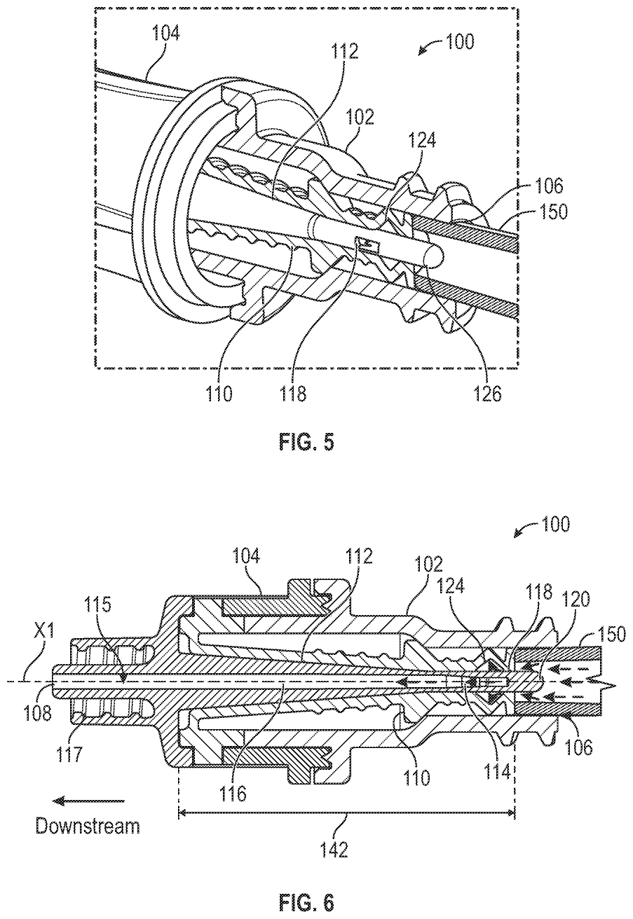

FIGS. 5 and 6 illustrate partial cross-sectional views of the connector assembly 100, further showing a fluid delivery device 150 inserted into the fluid inlet 106 of the housing 102 and engaging the valve 110, in accordance with some aspects of the present disclosure. The fluid inlet 106 of the housing 102 includes a size and shape that allows the fluid delivery device 150 to enter the housing 102 through the fluid inlet 106 and engage the valve 110. From the perspective of the valve 110, an external force is applied by the fluid delivery device 150. The external force provided by the fluid delivery device 150 causes displacement of the valve 110. For example, as shown in FIG. 6, the valve 110 compresses, or reduces to a dimension 142 (less than the dimension 140, shown in FIG. 4) based on the external force applied by the fluid delivery device 150. Also, the compression of the valve 110 represents relative movement of the valve 110 as compared to the housing 102, the housing 104, and the post 112.

Based on the displacement of the valve 110, the valve 110 is in the open position and can receive fluid from the fluid delivery device 150. Several arrows with dotted lines are shown in the connector assembly 100 and the fluid delivery device 150. The arrows represent fluid flow from the fluid delivery device 150 and through the connector assembly 100. Also, as shown in FIG. 6, the downstream direction of fluid flow is shown. In the open position of the valve 110, the distal end 120 of the post 112 protrudes through the slit 118 of the valve 110. Additionally, the displacement of the valve 110 exposes the opening 114 of the post 112. For purposes of illustration, the post 112 in FIG. 6 is rotated such that the opening 114 is viewable. When the opening 114 is uncovered by the valve 10, fluid flows from the fluid delivery device 150 through the slit 118 (now open) and subsequently to the lumen 116 by way of the opening 114. Accordingly, the slit 118 is fluidly connected to the opening 114 and the lumen 116.

As a result of the displacement of the valve 110, a fluid pocket 124 is formed in the valve 110. The fluid pocket 124 represents a volume, i.e., three-dimensional cavity or recess in the valve 110. In some embodiments, during fluid delivery provided by the fluid delivery device 150, the fluid pocket 124 receives at least some of the fluid. The volume of the fluid pocket 124 is dependent upon the amount of displacement of the valve 110 caused by the external force from the fluid delivery device 150 and may vary, as described below.

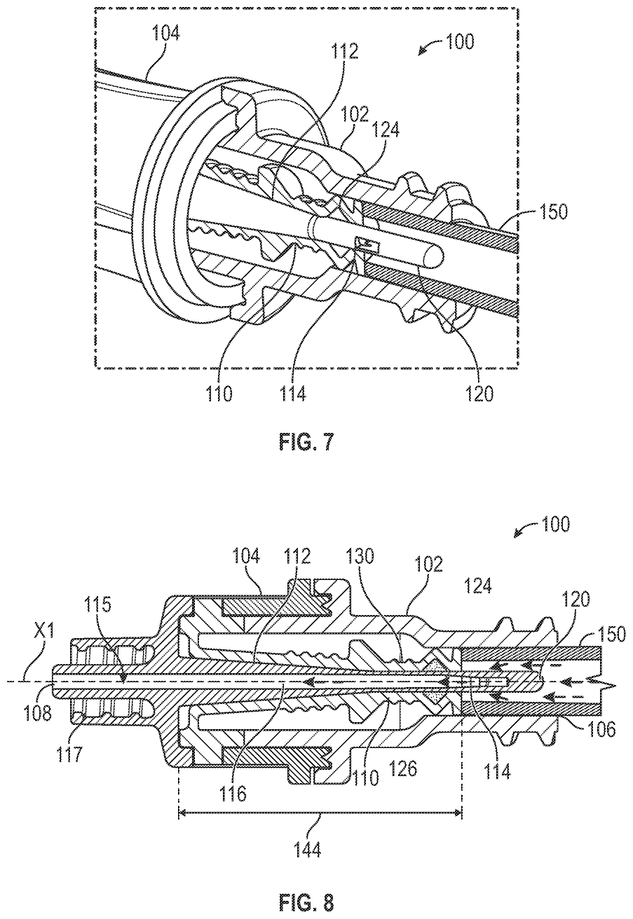

FIGS. 7 and 8 illustrate partial cross-sectional views of the connector assembly 100, showing the fluid delivery device 150 further inserted into the fluid inlet 106 of the housing 102 and further displacing the valve 110, in accordance with some aspects of the present disclosure. Based on the increased insertion into the fluid inlet 106 of the housing 102, the fluid delivery device 150 applies an additional external force to the valve 110, causing further displacement of the valve 110. For example, the valve 110 is further compressed within the housing 102, causing the distal end 120 of the post 112 to further protrude through the slit 118 of the valve 110. The valve 110 is further reduced to a dimension 144 (less than the dimension 142 of the valve 110, shown in FIG. 6). However, fluid provided by the fluid delivery device 150 can still be delivered through the opening 114 and into the lumen 116. For purposes of illustration, the post 112 in FIG. 8 is rotated such that the opening 114 is viewable.

Referring again to FIGS. 5 and 6, at least some fluid provided by the fluid delivery device 150 flows into the fluid pocket 124. However, based on the further compression of the valve 110 shown in FIGS. 7 and 8, the fluid pocket 124 is sealed off from the fluid path and the fluid pocket 124 is no longer fluidly connected to the fluid delivery device 150. As shown in FIG. 8, a fluid 126 is disposed in the fluid pocket 124. The fluid 126 represents residual fluid provided by the fluid delivery device 150 that is retained by the fluid pocket 124 prior to the fluid pocket 124 being sealed off from the fluid path. Also, the further compression of the valve 110 increases the volume of the fluid pocket 124, causing the valve 110 to engage an inner wall 130 of the housing 102 at a location of the valve 110 corresponding to the fluid pocket 124. In some embodiments, the engagement between the fluid pocket 124 of the valve 110 and the inner wall 130 of the housing 102 represents maximum compression of the valve 110 and maximum insertion of the fluid delivery device 150 into the fluid inlet 106 of the housing 102.

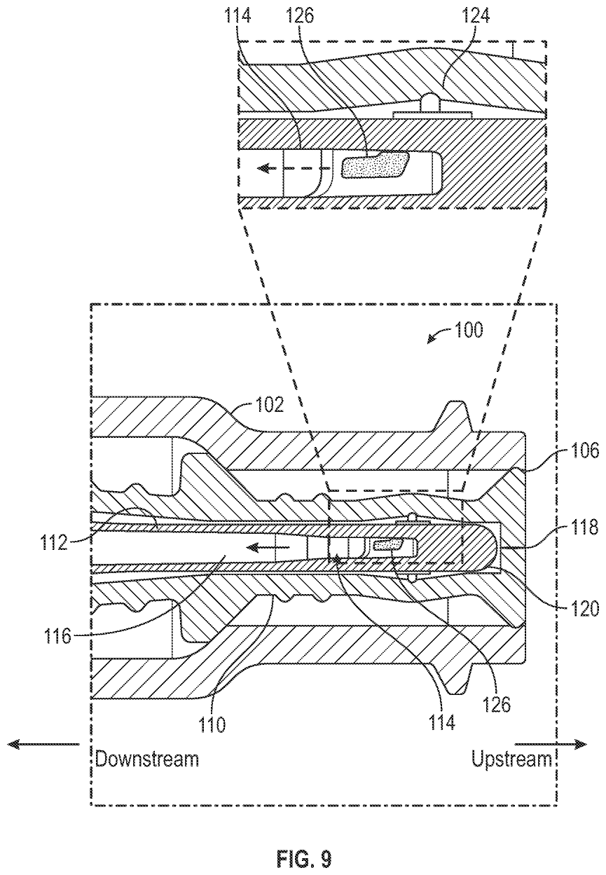

FIG. 9 illustrates a partial cross-sectional view of the connector assembly 100, with the fluid delivery device 150 removed from the housing 102, in accordance with some aspects of the present disclosure. As the fluid delivery device 150 (previously shown) is removed from the housing 102, the valve 110 begins to decompress. For example, the distal end 120 of the post 112 no longer protrudes through the slit 118 of the valve 110, and the valve 110 returns to the closed position. As a result, the valve 110 prevents fluid (external to the valve 110) from subsequently entering the valve 110. Accordingly, the valve 110 covers the opening 114 and the distal end 120 of the post 112. When the fluid delivery device 150 is removed from the fluid inlet 106 of the housing 102 and no longer engages the valve 110, the valve 110 is no longer displaced and returns to its original shape (shown in FIGS. 3 and 4). Although not shown, the valve 110 may cover the opening 114 prior to the fluid delivery device 150 being fully removed from the fluid inlet 106 of the housing 102 and prior to the fluid delivery device 150 being fully disengaged from the valve 110.

Also, due in part to the decompression of the valve 110, the fluid pocket 124 collapses. The collapsing action of the fluid pocket 124 drives the fluid 126 into the opening 114 of the post 112 and through the lumen 116. As shown in the enlarged view, the fluid 126 is driven out of the fluid pocket 124 based on the fluid pocket 124 collapsing. Moreover, the force provided by the collapsing of the fluid pocket 124 causes the fluid 126 to flow in the downstream direction, where the fluid 126 can enter a catheter line (not shown) connected to the connector assembly 100. Based on the collapsing of the fluid pocket 124 and the distal end 120 no longer protruding through the slit 118 of the valve 110 (thus closing the slit 118), forces provided by the closing of the valve 110 and/or the removal of the fluid delivery device 150 do not cause the fluid 126 to flow in the upstream direction, which is opposite to the downstream direction.

Also, the collapsing of the fluid pocket 124 changes the volume of the fluid pocket 124. In some embodiments, the collapsing reduces the volume of the fluid pocket 124 to a volume less than that when the valve 110 is displaced by the fluid delivery device 150. Also, in some embodiments, the lesser volume of the fluid pocket 124 in FIG. 9 includes a zero volume, indicating the fluid pocket 124 completely collapse to no volume.

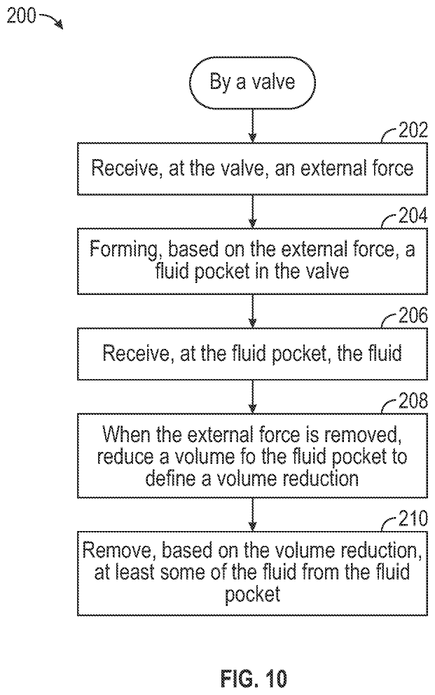

FIG. 10 illustrates a flowchart 200 showing a method for regulating a fluid to a catheter, in accordance with some aspects of the present disclosure. The method shown in the flowchart 200 may be performed by valves described herein. Accordingly, valves described herein can carry out the method shown in the flowchart 200.

In step 202, an external force is received at the valve. The external force may be provided by a fluid delivery device that delivers a fluid to a connector assembly that houses the valve. Based on the external force provided by the fluid delivery device, the valve may become displaced. The displacement may include a compression of at least a portion of the valve.

In step 204, a fluid pocket is formed in the valve based on the external force. The fluid pocket represents a recess or cavity in the valve when the valve is displaced. Additionally, due in part to the external force, the valve may open. Moreover, the external force may cause the valve to compress, further causing a post of the connector assembly to pass through a slit of the valve.

In step 206, fluid is received at the fluid pocket. The fluid received at the fluid pocket may include fluid that has yet to pass through the valve and to another structure, such as a catheter line.

In step 208, when the external force is removed, a volume of the fluid pocket is reduced and defines a volume reduction of the fluid pocket. For example, when the fluid pocket is formed based on the external force, the fluid pocket includes an initial volume. However, as the external force is removed, the valve begins to decompress and return to its initial form. As a result, the fluid pocket reduces in volume as the external force is removed. Moreover, in some embodiments, when the external force is fully removed, the fluid pocket reduces to zero volume.

In step 210, at least some of the fluid from the fluid pocket is removed based on the volume reduction. The volume reduction represents the fluid pocket collapsing as the external force is removed. Due to the reduced volume of the fluid pocket, the fluid is forced out of the fluid pocket. Further, the force provided by the collapsing fluid pocket can drive the fluid downstream through the valve and to the catheter line. Moreover, the valve can close and prevent upstream flow of the fluid.

Although the present disclosure includes embodiments in which a post includes a single opening in the Figures, it should be understood that the post may include any number of openings, each of each can receive a fluid from a fluid delivery device.

The features of the present disclosure provide multiple housings can be coupled together to form a fluid pathway therebetween. When coupled together, the features of the present disclosure resist unintentional separation between the housings. However, if the housings are separated, wither unintentionally or intentionally, the fluid pathway through the housing may become closed or obstructed to prevent fluid loss therefrom. The features of the present disclosure as provided that upon separation of the housings, any of the housings can be cleaned and disinfected, and the housings can be once again coupled together to form a fluid pathway therebetween.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A connector assembly for use with a catheter, the connector assembly comprising: a first housing comprising a fluid inlet; a second housing coupled with the first housing, the second housing comprising a post that includes an opening; and a valve that surrounds post, wherein: responsive to an external force, the valve comprises a fluid pocket, and when the external force is removed, the fluid pocket collapses and causes a fluid received from the fluid inlet and stored in the fluid pocket to enter the post through the opening.

Clause 2. The connector assembly of claim 1, wherein: an open position of the valve comprises the opening uncovered by the valve, and a closed position comprises the valve covering the opening.

Clause 3. The connector assembly of claim 2, wherein the external force causes the open position of the valve.

Clause 4. The connector assembly of claim 2, wherein: the valve comprises a first dimension based on the external force, and the valve comprises a second dimension when the external force is removed, the second dimension greater than the first dimension.

Clause 5. The connector assembly of claim 2, wherein: the fluid pocket is formed in the open position, and the fluid pocket collapses based on a transition of the valve from the open position to the closed position.

Clause 6. The connector assembly of claim 1, wherein: the first housing comprises an inner wall, and the fluid pocket contacts the inner wall.

Clause 7. The connector assembly of claim 1, wherein: the post comprises a distal end that extends into the first housing, an open position of the valve comprises the distal end uncovered by the valve, and a closed position of the valve comprises the valve covering the distal end.

Clause 8. The connector assembly of claim 1, wherein: the valve comprises a slit, and an open position of the valve comprises the post protruding through the slit.

Clause 9. A valve, comprising: a compressible body, wherein: responsive to an external force provided by a fluid delivery device, the compressible body reduces from a first dimension to a second dimension and forms a fluid pocket that receives a fluid from the fluid delivery device, and when the external force is removed, the compressible body increases from the second dimension to the first dimension and causes a volume reduction of the fluid pocket, the volume reduction causing the fluid to exit the fluid pocket.

Clause 10. The valve of claim 9, wherein: the external force opens the compressible body, thereby allowing the fluid from the fluid delivery device to flow in a first direction, and when the external force is removed, the compressible body closes, thereby preventing the fluid from flowing in a second direction opposite the first direction.

Clause 11. The valve of claim 9, wherein: the compressible body comprises a first dimension based on the external force, and the compressible body comprises a second dimension when the external force is removed, the second dimension greater than the first dimension.

Clause 12. The valve of claim 9, wherein: the external force causes the fluid pocket to define a first volume, and removal of the external forces causes the fluid pocket to define a second volume less than the first volume.

Clause 13. The valve of claim 9, wherein the compressible body comprises a slit that opens in response to the external force.

Clause 14. The valve of claim 13, wherein the slit closes when the external force is removed.

Clause 15. 15. The valve of claim 13, wherein the compressible body comprises a channel that receives a post, and responsive to the external force the slit opens and is fluidly connected to the channel.

Clause 16. A method for regulating a fluid to a catheter, the method comprising, by a valve: receiving, at the valve, an external force; forming, based on the external force, a fluid pocket in the valve; receiving, at the fluid pocket, the fluid; when the external force is removed, reducing a volume of the fluid pocket to define a volume reduction; and removing, based on the volume reduction, at least some of the fluid from the fluid pocket.

Clause 17. The method of claim 16, wherein when the external force is removed, the fluid pocket collapses and causes at least some of the fluid to enter a channel of the valve.

Clause 18. The method of claim 17, driving, based on the volume reduction, at least some of the fluid downstream through the valve.

Clause 19. The method of claim 16, further comprising: prior to receiving the fluid at the fluid pocket, receiving, at a slit formed in the valve, the fluid; and when the external force is removed, driving the fluid downstream such that the fluid is prevent from passing through the slit.

Clause 20. The method of claim 16, wherein receiving the external force comprising receiving contact from a fluid delivery device.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A connector assembly for use with a catheter, the connector assembly comprising:
a first housing comprising a fluid inlet;
a second housing coupled with the first housing, the second housing comprising a post that includes an opening; and
a valve that surrounds post, wherein:
responsive to an external force, the valve comprises a fluid pocket, and
when the external force is removed, the fluid pocket collapses and causes a fluid received from the fluid inlet and stored in the fluid pocket to enter the post through the opening.

2. The connector assembly of claim 1, wherein:
an open position of the valve comprises the opening uncovered by the valve, and a closed position comprises the valve covering the opening.

3. The connector assembly of claim 2, wherein the external force causes the open position of the valve.

4. The connector assembly of claim 2, wherein the valve comprises a first dimension based on the external force, and the valve comprises a second dimension when the external force is removed, the second dimension greater than the first dimension.

5. The connector assembly of claim 2, wherein the fluid pocket is formed in the open position, and the fluid pocket collapses based on a transition of the valve from the open position to the closed position.

6. The connector assembly of claim 1, wherein the first housing comprises an inner wall, and the fluid pocket contacts the inner wall.

7. The connector assembly of claim 1, wherein the post comprises a distal end that extends into the first housing, and an open position of the valve comprises the distal end uncovered by the valve, and a closed position of the valve comprises the valve covering the distal end.

8. The connector assembly of claim 1, wherein the valve comprises a slit, and
an open position of the valve comprises the post protruding through the slit.

9. A valve, comprising:
a compressible body, wherein:
responsive to an external force provided by a fluid delivery device, the compressible body reduces from a first dimension to a second dimension and forms a fluid pocket that receives a fluid from the fluid delivery device, and
when the external force is removed, the compressible body increases from the second dimension to the first dimension and causes a volume reduction of the fluid pocket, the volume reduction causing the fluid to exit the fluid pocket.

10. The valve of claim 9, wherein the external force opens the compressible body, thereby allowing the fluid from the fluid delivery device to flow in a first direction, and when the external force is removed, the compressible body closes, thereby preventing the fluid from flowing in a second direction opposite the first direction.

11. The valve of claim 9, wherein the compressible body comprises a first dimension based on the external force, and the compressible body comprises a second dimension when the external force is removed, the second dimension greater than the first dimension.

12. The valve of claim 9, wherein the external force causes the fluid pocket to define a first volume, and removal of the external forces causes the fluid pocket to define a second volume less than the first volume.

13. The valve of claim 9, wherein the compressible body comprises a slit that opens in response to the external force.

14. The valve of claim 13, wherein the slit closes when the external force is removed.

15. The valve of claim 13, wherein the compressible body comprises a channel that receives a post, and responsive to the external force the slit opens and is fluidly connected to the channel.

16. A method for regulating a fluid to a catheter, the method comprising, by a valve:
receiving, at the valve, an external force;
forming, based on the external force, a fluid pocket in the valve;
receiving, at the fluid pocket, the fluid;
when the external force is removed, reducing a volume of the fluid pocket to define a volume reduction; and
removing, based on the volume reduction, at least some of the fluid from the fluid pocket.

17. The method of claim 16, wherein when the external force is removed, the fluid pocket collapses and causes at least some of the fluid to enter a channel of the valve.

18. The method of claim 17, driving, based on the volume reduction, at least some of the fluid downstream through the valve.

19. The method of claim 16, further comprising:
prior to receiving the fluid at the fluid pocket, receiving, at a slit formed in the valve, the fluid; and
when the external force is removed, driving the fluid downstream such that the fluid is prevent from passing through the slit.

20. The method of claim 16, wherein receiving the external force comprising receiving contact from a fluid delivery device.
